(19) United States
(12) Patent Application Publication
(10) Pub. No.: US 2021/0401879 A1
(43) Pub. Date: Dec. 30, 2021

(54) COMPOSITIONS FOR CONTROLLING ODOR AND ITCH AND METHODS OF AND DEVICES FOR ADMINISTERING SAME

(71) Applicant: Vireo Systems, Inc., Madison, TN (US)

(72) Inventors: Mark C. Faulkner, Madison, TN (US); Deeannah Seymour, Franklin, TN (US)

(73) Assignee: Vireo Systems, Inc., Madison, TN (US)

(21) Appl. No.: 17/364,105

(22) Filed: Jun. 30, 2021

Related U.S. Application Data
(60) Provisional application No. 63/045,891, filed on Jun. 30, 2020.

(51) Int. Cl. A61K 33/22; A61K 9/10; A61K 9/00; A61K 47/44

(52) U.S. Cl. CPC A61K 33/22; A61K 47/44; A61K 9/0034; A61K 9/10

(57) ABSTRACT

Compositions, such as aqueous solutions and non-aqueous solutions and slurries, of a weak acid are provided. The disclosed compositions may be used to treat, prevent, and/or reduce one or more symptoms associated with numerous vaginal conditions or disturbances, such as odor and itch.

### FIELD OF THE INVENTION

The present invention generally relates to compositions of a weak acid, such as solutions and slurries, and their use to treat or reduce one or more symptoms associated with numerous vaginal conditions such as odor and itch. The present invention also relates to methods of administering the compositions of the present invention and devices for use in such administration.

### BACKGROUND OF THE INVENTION

Throughout a female's life, the vaginal ecosystem experiences instability (acute and chronic) caused by human behavior and/or the use of vaginal products and contraceptives. If the vaginal ecosystem cannot naturally recover from these periods of instability, then various levels of discomfort (itching, burning, irritation) may occur. For example, vaginitis, which is generally described to be inflammation of the vulvovaginal region, may include symptoms such as change in color, odor, or amount of discharge, vaginal itching, burning, or general irritation. The three major causes of this inflammation include vaginal candidiasis, bacterial vaginosis and trichomonas. If not treated, vaginitis can lead to more serious medical problems.

Boric acid, when vaginally administered, acts as a bacteriostatic and fungistatic agent. For example, U.S. Pat. No. 10,258,567 describes a probiotic vaginal formulation that includes boric acid, a carrier, and a probiotic that is administered as a powder with an applicator or suppository for treating or preventing urogenital infections and inflammatory conditions, including vaginitis. In addition, U.S. Pat. No. 6,420,425 generally describes compositions including a combination of boric acid and acetic acid administered to a patient for the treatment and prevention of vaginal and skin infections. Boric acid has also been used as an odor neutralizer in personal absorbent articles. For instance, European Patent No. 1,575,628 describes the use of odor control agents, including boric acid, in feminine products, diapers, and the like. Other hygiene articles have been described to use odor control compositions including an organic zinc salt and an anti-microbial agent such as an essential oil of thyme or thymol (see, e.g., U.S. Pat. No. 10,517,983).

Vaginal douches have been proposed for reducing vaginal odors. Conventional vaginal douches, which typically involve the application of a stream of douching fluid through a vaginal douche applicator and into the vaginal canal of the user, usually employ applicators made from inexpensive materials, such as plastic, intended to be disposed after a single use, but rendering it difficult to release all of the douching fluid into the vaginal canal. Moreover, the douching fluid tends not to be effective in alleviating some vaginal odors or may serve only to temporarily mask odor. In addition, the douching fluid may alter the natural pH (about 4.0 to 4.5) and/or otherwise irritate the vaginal canal. While a vinegar-water solution closely mimics the normal pH of the vaginal canal, such a solution, if effective against odor, only lasts for a short period of time. U.S. Pat. No. 7,276,056 purportedly discloses a device and method to overcome the deficiencies in conventional douches where contact of the stainless-steel external surfaces of the douche applicator with the vaginal tissue in the presence of water causes ionization or chemical reactions with odor linked chemical bonds that results in breakage of the odor linked chemical bonds and neutralization of their odor carrying capabilities. However, use of such a device may be uncomfortable and/or ineffective if not properly employed.

There remains a need in the art for safe, effective, and easy to administer compositions for use at home to alleviate and/or eliminate symptoms of vaginitis. In addition, it would be advantageous if such composition normalizes and stabilizes vaginal disturbances. Moreover, it would be advantageous to arrive at such a composition for use in combination with other medicaments that treat vaginal candidiasis, bacterial vaginosis and/or trichomonas. The present invention addresses such needs.

### SUMMARY OF THE INVENTION

The problems expounded above, as well as others, are addressed by the following inventions, although it is to be understood that not every embodiment of the inventions described herein will address each of the problems described above.

In some embodiments, an aqueous solution for alleviating at least one of odor and vaginal itch in a subject is provided, the aqueous solution including a weak acid having a pH of about 3 to about 7 uniformly distributed in a solvent comprising water, wherein the aqueous solution has a pH of about 2 to about 7, and wherein the weak acid is included in the aqueous solution in an amount of about 5 mg/mL to about 8 mg/mL. In one embodiment, the weak acid is selected from the group consisting of boric acid, hydrogen sulfide, ethanoic/acetic acid, lactic acid, citric acid, tartaric acid, ascorbic acid, maleic acid, propionic acid, carbonic acid, and combinations thereof. For example, the weak acid is boric acid. In another embodiment, the boric acid is included in the aqueous solution in an amount of about 6 mg/mL to about 7 mg/mL. In still another embodiment, the aqueous solution may have a molar concentration of about 50 mol/L to about 150 mol/L.

In other embodiments, a method for alleviating at least one of odor and vaginal itch in a subject is provided, the method including vaginally administering to the subject an effective amount of an aqueous solution including a weak acid having a pH of about 3 to about 7 uniformly distributed in a solvent including water, wherein the aqueous solution has a pH of about 2 to about 7, and wherein the weak acid is included in the aqueous solution in an amount of about 5 mg/mL to about 8 mg/mL. In one embodiment, the weak acid is selected from the group consisting of boric acid, hydrogen sulfide, ethanoic/acetic acid, lactic acid, citric acid, tartaric acid, ascorbic acid, maleic acid, propionic acid, carbonic acid, and combinations thereof. For example, the weak acid is boric acid. In another embodiment, the aqueous solution is administered to the subject in the form of a vaginal device configured for insertion into the vagina. For instance, the aqueous solution may be administered to the subject in the form of a bellows bottle. In still another embodiment, the aqueous solution may be administered to the subject in the form of a vaginal douche. In yet another embodiment, the boric acid is included in the aqueous solution in an amount of about 6 mg/mL to about 7 mg/mL.

In still other embodiments, a vaginal capsule is provided, the vaginal capsule including an effective amount of a slurry including a weak acid having a pH of about 3 to about 7 dispersed in a dispersing medium, and wherein the weak acid is included in the slurry in an amount of about 250 mg/mL to about 700 mg/mL. In this embodiment, the weak acid is selected from the group consisting of boric acid, hydrogen sulfide, ethanoic/acetic acid, lactic acid, citric acid, tartaric acid, ascorbic acid, maleic acid, propionic acid, carbonic acid, and combinations thereof. For instance, the weak acid is boric acid. In another embodiment, the dispersing medium is selected from the group consisting of cocoa butter, cottonseed oil, lipid nanoparticles, coconut oil, palm oil, palm kernel oil, apricot kernel oil, argan oil, baobab seed oil, calendula oil, grapeseed oil, jojoba oil, sesame seed oil, shea butter, sunflower oil, almond oil, wheat germ oil, and combinations thereof. For example, the dispersing medium is coconut oil. In still another embodiment, the slurry further includes an antioxidant selected from the group consisting of vitamin E, vitamin A, vitamin C, hyaluronic acid, collagen, glucosamine sulfate, chondroitin sulfate, alpha-lipoic acid, and combinations thereof. For instance, the slurry further includes vitamin E. In yet another embodiment, the vaginal capsule is a gelatin capsule. The vaginal capsule may also have one or more controlled release outer coatings.

In yet other embodiments, a vaginal suppository is provided, the vaginal suppository including an effective amount of a slurry including boric acid dispersed in a dispersing medium including coconut oil, and wherein the boric acid is included in the slurry in an amount of about 200 mg/mL to about 800 mg/mL. In one embodiment, the slurry further includes an antioxidant selected from the group consisting of vitamin E, vitamin A, vitamin C, hyaluronic acid, collagen, glucosamine sulfate, chondroitin sulfate, alpha-lipoic acid, and combinations thereof. For example, the slurry further includes vitamin E, ascorbic acid (vitamin C), and combinations thereof. In another embodiment, the antioxidant is present in the slurry in an amount of about 5 percent to about 25 percent by weight of the slurry. In still another embodiment, the dispersing medium is present in the slurry in an amount of about 1 mL to about 5 mL. The vaginal suppository may have one or more controlled release outer coatings.

In some embodiments, a method for alleviating at least one of odor and vaginal itch in a subject is provided, the method including vaginally administering to the subject an effective amount of a slurry including a weak acid having a pH of about 3 to about 7 dispersed in a dispersing medium, and wherein the weak acid is included in the slurry in an amount of about 200 mg/mL to about 800 mg/mL. In this embodiment, the weak acid is selected from the group consisting of boric acid, hydrogen sulfide, ethanoic/acetic acid, lactic acid, citric acid, tartaric acid, ascorbic acid, maleic acid, propionic acid, carbonic acid, and combinations thereof. For example, the weak acid is boric acid. In another embodiment, the slurry is administered to the subject in the form of a vaginal capsule. In still another embodiment, the slurry is administered to the subject in the form of a vaginal suppository. In yet another embodiment, the dispersing medium is selected from the group consisting of cocoa butter, cottonseed oil, lipid nanoparticles, coconut oil, palm oil, palm kernel oil, apricot kernel oil, argan oil, baobab seed oil, calendula oil, grapeseed oil, jojoba oil, sesame seed oil, shea butter, sunflower oil, almond oil, wheat germ oil, and combinations thereof. For example, the dispersing medium may be coconut oil. The slurry may further include an antioxidant selected from the group consisting of vitamin E, vitamin A, vitamin C, hyaluronic acid, collagen, glucosamine sulfate, chondroitin sulfate, alpha-lipoic acid, and combinations thereof.

In other embodiments, a method of adjusting pH in a mammalian vagina is provided, the method including administering an effective amount of a composition to a mammalian vagina having a first pH greater than about 4.5, wherein the composition includes a weak acid having a pH of about 3 to about 7, and wherein the effective amount is sufficient to reduce the first pH to a second pH of about 4.5 or less. In this embodiment, the mammalian vagina is a human vagina. In another embodiment, the composition is formulated as an aqueous solution including the weak acid uniformly distributed in a solvent including water. In still another embodiment, the composition is formulated as a slurry including the weak acid dispersed in a dispersing medium including cocoa butter, cottonseed oil, lipid nanoparticles, coconut oil, palm oil, palm kernel oil, apricot kernel oil, argan oil, baobab seed oil, calendula oil, grapeseed oil, jojoba oil, sesame seed oil, shea butter, sunflower oil, almond oil, wheat germ oil, or combinations thereof. The slurry may be administered to the mammalian vagina in the form of a vaginal capsule or vaginal suppository. In yet another embodiment, the weak acid is in powder form and is administered to the mammalian vagina in the form of a vaginal capsule or vaginal suppository. In another embodiment, the effective amount is sufficient to reduce the first pH to a second pH that is at least about 0.5 lower than the first pH, for example, at least about 1.0 lower than the first pH.

In still other embodiments, a method of adjusting pH in a mammalian vagina is provided, the method including administering an effective amount of a composition to a mammalian vagina, such as a human vagina, having a first pH greater than about 4.5 and including odoriferous amines, wherein the composition includes boric acid, and wherein the effective amount is sufficient to (i) reduce the first pH to a second pH of about 4.5 or less and (ii) cause protonation of the odoriferous amines. In one embodiment, the composition is formulated as a slurry including the boric acid dispersed in a dispersing medium including coconut oil. In another embodiment, the composition is formulated as an aqueous solution including the boric acid uniformly distributed in water. In still another embodiment, the effective amount is sufficient to reduce the first pH to a second pH of about 4.0. In yet another embodiment, the effective amount is sufficient to reduce the first pH to a second pH that is at least about 2.0 lower than the first pH.

In yet other embodiments, a method of adjusting pH in a mammalian vagina is provided, the method including administering an effective amount of a composition to a mammalian vagina having a first pH greater than about 4.5, wherein the composition includes a weak acid having a pKa of about 7 to about 9.5, and wherein the effective amount is sufficient to reduce the first pH to a second pH of about 4.5 or less.

### BRIEF DESCRIPTION OF THE DRAWINGS

Further features and advantages of the invention can be ascertained from the following detailed description that is provided in connection with the drawing(s) described below:

FIG. 1 is a frontal view of a portion of a device for administering a vaginal solution in accordance with one embodiment of the present invention.

FIG. 2 is a frontal view of a portion of a device for administering a vaginal solution in accordance with one embodiment of the present invention.

FIG. 3A is a graph showing the percentage of subjects having a certain vaginal pH before treatment with a vaginal suppository containing a non-aqueous slurry according to an embodiment of the present disclosure.

FIG. 3B is a graph showing the percentage of subjects having a certain vaginal pH after treatment with a vaginal suppository containing a non-aqueous slurry according to an embodiment of the present disclosure.

FIG. 4A is a graph showing the percentage of subjects having a certain vaginal pH before treatment with a vaginal suppository containing a composition according to another embodiment of the present disclosure.

FIG. 4B is a graph showing the percentage of subjects having a certain vaginal pH after treatment with a vaginal suppository containing a composition according to another embodiment of the present disclosure.

### DETAILED DESCRIPTION OF THE INVENTION

The present invention is directed to compositions, such as aqueous and non-aqueous solutions and slurries, of a weak acid useful for treating or reducing one or more symptoms associated with numerous vaginal conditions, and methods and apparatuses for administering same. More specifically, the vaginal compositions of the present invention are believed to reduce or eliminate vaginal odor, vaginal discomfort, itchiness, redness, and/or burning in and around the vagina. Moreover, without being bound to any particular theory, the vaginal compositions are believed to aid in restoring the vagina's natural pH balance and boost its natural cleansing system. Indeed, since the amount of the weak acid used is relatively low compared to treatments specifically targeted to kill vaginal bacteria, it may not be sufficient to completely restore the vagina's natural pH balance, but it may aid in doing so. Finally, the disclosed compositions and methods of administering also provide faster relief of the targeted symptoms when compared to or combined with conventional treatments, such as the use of antibiotics like metronidazole, clindamycin, and tinidazole.

Prior to setting forth this disclosure in more detail, it may be helpful to an understanding thereof to provide definitions of certain terms to be used herein. Additional definitions are set forth throughout this disclosure.

### Definitions

Unless otherwise defined, all terms (including technical and scientific terms) used herein have the same meaning as commonly understood by one of ordinary skill in the art of this disclosure. It will be further understood that terms, such as those defined in commonly used dictionaries, should be interpreted as having a meaning that is consistent with their meaning in the context of the specification and should not be interpreted in an idealized or overly formal sense unless expressly so defined herein. Well-known functions or constructions may not be described in detail for brevity or clarity.

As used herein, the singular forms “a,” “an,” and “the” include plural referents unless the context clearly dictates otherwise. For example, reference to “the method of treatment” includes reference to equivalent steps and methods known to those skilled in the art, and so forth.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other embodiments, the values may range in value either above or below the stated value in a range of approx. +/−5%; in other embodiments, the values may range in value either above or below the stated value in a range of approximately +/−2%; in other embodiments, the values may range in value either above or below the stated value in a range of approximately +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The terms “administering” or “administration” include acts such as prescribing, dispensing, giving, or applying a substance such that what is prescribed, dispensed, given, or applied actually contacts the patient's body externally or internally (or both). It is specifically contemplated that instructions or a prescription by a medical professional to a subject or patient to take or otherwise self-administer a substance is an act of administration.

The terms “alleviate” or “alleviation” refer to a lessening of the severity of one or more symptoms.

The term “Amsel's criteria” refers to the diagnostic criteria used to identify bacterial vaginosis (BV). These criteria include: homogenous white discharge that smoothly coats the vaginal mucosa; presence of clue cells (bacteria adhering to the vaginal epithelial cells, >20 percent of total cells on wet mount); vaginal pH of >4.5; positive whiff test (fishy odor with addition of 10 percent potassium hydroxide (KOH) to vaginal fluid). In order for a clinical diagnosis of BV, three of the four Amsel's criteria must be present.

The terms “effective amount” or “therapeutically effective amount” as used herein refer to an amount of a component or agent, either alone or as a part of a composition or solution, that is capable of having any detectable, positive effect on any symptom, aspect, or characteristics of an infection or condition. Such effect need not be absolute to be beneficial.

The terms “in need of treatment” and “in need of prevention” as used herein refer to a judgment made by a decision maker that a patient requires or will benefit from treatment or prevention. This judgment is made based on a variety of factors that are in the realm of a decision maker's expertise, but that includes the knowledge that the patient is suffering from symptoms, or is susceptible to symptoms, as the result of a condition that is treatable by a method or composition of the present disclosure. In this respect, the decision maker may be the individual suffering from symptoms or susceptible to symptoms (i.e., the patient herself) or a caregiver having knowledge of the patient's symptoms or susceptibility thereto.

The terms “individual,” “subject,” and “patient” are used interchangeably herein, and refer to any animal, including mammals, such as mice, rats, other rodents, rabbits, dogs, cats, swine, cattle, sheep, horses, primates, and humans.

The terms “inhibit,” “decrease,” and/or “reduce the likelihood of” (and like terms) generally refers to the act of reducing, either directly or indirectly, a function, activity, or behavior relative to the natural, expected, or average or relative to current conditions. It is understood that this is typically in relation to some standard or expected value, in other words it is relative, but that it is not always necessary for the standard or relative value to be referred to. Such terms can include complete inhibition, complete reduction, or elimination of the likelihood of a function, activity, or behavior relative to the natural, expected, or average or relative to current conditions.

The term “Nugent Score” refers to a weighted score between 0 and 10 derived from a microbiological analysis using a Gram-stained vaginal smear (Nugent et al., 1991, J. Clin. Microbiol., 29(2): 297-301). While a Nugent Score of 0-3 is considered normal, a score of 4-6 (intermediate) is indicative of a disrupted vaginal microenvironment, and 7-10 is defined as BV.

The terms “prevention,” “prevent,” “preventing,” “suppression,” “suppress,” and “suppressing” as used herein refer to a course of action initiated prior to the onset of a clinical manifestation of an infection or condition so as to reduce the likelihood or severity of such clinical manifestation of the infection or condition. Such reduction of the likelihood or severity need not be absolute to be useful. The terms also refer to inhibiting the full development of an infection or condition in a subject who is at risk of developing the disease state or condition.

The terms “treat,” “treating,” or “treatment” refers to at least partially attaining a desired therapeutic outcome. The therapeutic outcome may be the alleviation, reduction, or inhibition of one or more symptoms of vaginal infections such as the alleviation of vaginal odor, itch, or discharge or may be the reduction or normalization (or maintenance) of certain diagnostic criteria for vaginal infections such as vaginal pH, presence of clue cells, or a positive whiff test as defined by the Amsel criteria, by achieving normal Nugent Score of 0-3, or by determining through quantitative bacterial PCR that M. hominis, G. vaginali, and Lactobacillus bacterial counts normalized, i.e., as compared to the original PCR that detected the presence of BV through significant increases in M. hominis and G. vaginalis bacterial counts and significant decreases in Lactobacillus bacterial counts (see, e.g., Zozaya-Hinchliffe M, Lillis R, Martin D H, Ferris M J. Quantitative PCR assessments of bacterial species in women with and without bacterial vaginosis. J Clin Microbiol. 2010; 48(5):1812-1819; Sha B E, Chen H Y, Wang Q J, Zariffard M R, Cohen M H, Spear G T. Utility of Amsel criteria, Nugent score, and quantitative PCR for Gardnerella vaginalis, Mycoplasma hominis, and Lactobacillus spp. for diagnosis of bacterial vaginosis in human immunodeficiency virus-infected women. J Clin Microbiol. 2005; 43(9):4607-4612).

The term “vaginal solution” refers to a solution of this disclosure intended for delivery to affected subjects, e.g., humans. For example, a vaginal solution of the present disclosure may be formulated or used as a stand-alone treatment, an over-the-counter (OTC) medicament, a botanical drug, an herbal medicine, a cosmetic, a homeopathic agent, or any other form of health care product, optionally reviewed and approved by a government agency.

The term “vaginal slurry” refers to a colloid or suspension of this disclosure intended for delivery to affected subjects, e.g., humans. For example, a vaginal slurry of the present disclosure may be formulated or used as a stand-alone treatment, an over-the-counter (OTC) medicament, a botanical drug, an herbal medicine, a cosmetic, a homeopathic agent, or any other form of health care product, optionally reviewed and approved by a government agency.

### Compositions

The compositions of the invention include one or more weak acids. As used herein, the term “weak acid” refers to an acid that is partially dissociated into its ions in an aqueous solution or water and has a pH value at 1 mM of less than 7. In one embodiment, the weak acid has a pH at 1 mM of about 2 to less than about 6.5. In another embodiment, the weak acid has a pH at 1 mM of about 3 to about 6.3. In still another embodiment, the weak acid has a pH at 1 mM of about 3 to about 6.2. In yet another embodiment, the pH of the weak acid at 1 mM is about 3.05 to about 6.15. For example, the pH of the weak acid at 1 mM is about 3.5 to 6.2. In one embodiment, the weak acid may also have a pKa value in the range of about 4 to about 9.5. In another embodiment, the weak acid has a pKa value in the range of about 7 to about 9.5. For example, the weak acid may have a pH at 1 mM of about 4.9 to about 6.12 and a pKa value of about 7 to about 9.3.

Suitable weak acids contemplated by the present disclosure include, but are not limited to, boric acid, hydrogen sulfide, ethanoic/acetic acid, lactic acid, citric acid, tartaric acid, ascorbic acid, maleic acid, propionic acid, carbonic acid, and combinations thereof.

In some embodiments, the disclosed compositions include boric acid as the weak acid. Boric acid, also known as hydrogen borate, is a weak, tribasic Lewis acid of boron having the structure of formula (II):

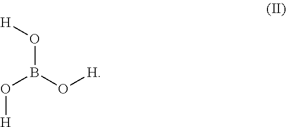

The boric acid may be produced by any suitable method known to one of ordinary skill in the art. In one embodiment, the boric acid is prepared by treating borax with nitric acid in accordance with the following:

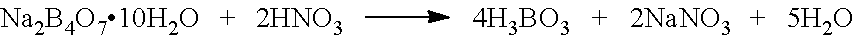

For example, borax (Na₂B₄O₇.10H₂O) may be dissolved in boiling distilled water and filtered. An excess of nitric acid is then added to the hot filtrate. After crystals of boric acid form, they are collected by filtration and washed with cold distilled water. The boric acid may then be purified by redissolving the washed crystals in boiling distilled water. In another embodiment, boric acid is prepared by treating borax with hydrochloric acid.

In other embodiments, the compositions of the present invention include propionic acid as the weak acid. The propionic acid may be produced by any suitable method known to those of ordinary skill in the art. For example, the propionic acid may be prepared by hydrocarboxylation of ethylene using nickel carbonyl as catalyst or aerobic oxidation of propionaldehyde. In still other embodiments, the compositions of the present invention include carbonic acid, which may be produced by any suitable method known to those of ordinary skill in the art. For example, the carbonic acid may be prepared by reacting calcium carbonate and hydrochloric acid in the presence of water.

The compositions of the present invention may include two or more weak acids. For example, in this aspect, the solution may include boric acid and a second weak acid, e.g., citric acid. In still another embodiment, the solution includes three or more weak acids.

### Aqueous Solutions

In some embodiments, the compositions of the present invention are in the form of a vaginal aqueous solution with the one or more weak acids uniformly distributed in a solvent. In one embodiment, the solvent is water. In other embodiments, the solvent may be an aqueous solution including ethanol, methanol, propylene glycol, polyethylene glycol (400 and 1600), sorbitol, mannitol, pyridine, and glycerin. The aqueous solutions may also include cocoa butter, cottonseed oil, lipid nanoparticles, glycerides and/or triglycerides of saturated fatty acids such as coconut, palm, and palm kernel oils.

The aqueous solution may contain about 1 mg/mL to about 12 mg/mL weak acid. In one embodiment, the weak acid is included in an amount of about 2 mg/mL to about 11.4 mg/mL. In another embodiment, the weak acid is included in the aqueous solution in an amount of about 4 mg/mL to about 9 mg/mL. In yet another embodiment, the aqueous solution includes about 5 mg/mL to about 8 mg/mL of weak acid. In still another embodiment, the weak acid is included in the aqueous solution in an amount of about 6 mg/mL to about 7 mg/mL. For example, the aqueous solution may contain about 6 mg/mL to about 7 mg/mL of boric acid.

In some embodiments, the resultant aqueous solution has a molar concentration of about 50 mol/L to about 150 mol/L. In one embodiment, the resultant aqueous solution has a molar concentration of about 75 mol/L to about 120 mol/L. In another embodiment, the resultant aqueous solution has a molar concentration of about 80 mol/L to about 110 mol/L.

The pH of the resultant aqueous solution may be about 7 or below at about 20° C. In another embodiment, the resultant aqueous solution may have a pH of about 2 to about 7 at about 20° C. In yet another embodiment, the pH of the resultant aqueous solution is from about 2 to about 6.5. In still another embodiment, the pH of the resultant aqueous solution is from about 2.5 to about 7. For example, the resultant aqueous solution may have a pH of from about 2.5 to about 6.5. In yet another embodiment, the pH of the resultant aqueous solution is from about 4.5 to about 7. In still another embodiment, the pH of the resultant aqueous solution is from about 5.0 to about 6.

The aqueous solutions of the present invention may also include a preservative. Suitable preservatives include, but are not limited to, hydrogen peroxide, polymeric quaternary ammonium compounds, chlorine containing preservatives such as benzalkonium chloride, and combinations thereof. In one embodiment, the solutions of the present invention are substantially free of any chloride containing preservatives and, particularly, are substantially free of benzalkonium chloride.

### Non Aqueous Solutions and Slurries

In other embodiments, the compositions of the present invention are in the form of a vaginal non-aqueous solution with the one or more weak acids uniformly distributed in a dispersing medium. In another embodiment, the compositions are in the form of a vaginal slurry with the one or more weak acids dispersed in a dispersing medium.

The dispersing medium may be a carrier dispersing medium. Suitable dispersing media contemplated by the present invention include, but are not limited to, cocoa butter, cottonseed oil, lipid nanoparticles, glycerides and/or triglycerides of saturated fatty acids, such as coconut, palm, and palm kernel oils, apricot kernel oil, argan oil, baobab seed oil, calendula oil, grapeseed oil, jojoba oil, sesame seed oil, shea butter, sunflower oil, almond oil, wheat germ oil, and combinations thereof.

In some embodiments, the dispersing medium is coconut oil. Coconut oil is a moisturizer that helps keep the skin moist and protected by trapping moisture in the skin. The moisturizing properties of coconut oil are advantageous for keeping the vagina moist and preventing vaginal dryness. In one embodiment, the dispersing medium is refined coconut oil. In another embodiment, the dispersing medium is a fractionated coconut oil. In particular, fractionated coconut oil (or Medium Chain Triglycerides (MCT) oil) is a suitable dispersing medium for use with the present invention because the oil stays liquid at temperatures down to −20° F. and is highly stable with an indefinite shelf life. In this aspect, the MCT oil includes the capric and caprylic acids, but the long chain triglycerides have been removed.

In another embodiment, the dispersing medium is a blend of at least two of the above carrier dispersing mediums. For example, the dispersing medium may include a blend of argan oil and calendula oil, a blend of grapeseed oil and almond oil, a blend of coconut oil and grapeseed oil, and other beneficial blends from the carrier dispersing mediums described above. Without being bound to any particular theory, blending two or more carrier dispersing mediums will enhance the anti-inflammatory, anti-bacterial, and antioxidant properties of the solutions of the present invention. In still another embodiment, the dispersing medium is a blend of fractionated coconut oil, sesame seed oil, grapeseed oil, almond oil, sunflower oil, and wheat germ oil.

In another embodiment, the dispersing medium is hydrogenated castor oil. In another embodiment, the dispersing medium includes ethanol, methanol, propylene glycol, polyethylene glycol (400 and 1600), sorbitol, mannitol, pyridine, glycerin, or a combination thereof. In still another embodiment, the dispersing medium includes lecithin, aluminum stearate, an oil insoluble excipient such as sucrose, or a combination thereof.

The dispersing medium may be a bio-based dispersing medium including, but not limited to, those dispersing mediums derived from agricultural crops such as corn. In one embodiment, the dispersing medium is ethyl lactate.

The non-aqueous solutions and slurries of the present invention may also include one or more antioxidants. Suitable antioxidants include, but are not limited to, vitamin E, vitamin A, hyaluronic acid, collagen, glucosamine sulfate, chondroitin sulfate, alpha-lipoic acid, and combinations thereof. In one embodiment, the non-aqueous solutions and slurries of the present invention include vitamin E. In this embodiment, vitamin E may be included as DL-alpha-tocopheryl acetate, which may optionally be in a microencapsulated form. The non-aqueous solutions and slurries of the present invention may include about 5 percent to about 25 percent by weight of the antioxidant, such as vitamin E. In one embodiment, the antioxidant, such as vitamin E, may be present in the non-aqueous solutions and slurries in an amount of about 8 percent to about 20 percent by weight of the solution.

The non-aqueous solutions and slurries of the present invention may also include a preservative. Suitable preservatives include, but are not limited to, hydrogen peroxide, polymeric quaternary ammonium compounds, chlorine containing preservatives such as benzalkonium chloride, and combinations thereof. In one embodiment, the non-aqueous solutions and slurries of the present invention are substantially free of any chloride containing preservatives and, particularly, are substantially free of benzalkonium chloride.

Also, when not included as the weak acid, ascorbic acid may also be used as an antioxidant and preservative. In this aspect, the ascorbic acid may be present in an amount of about 5 to about 25 percent by weight of the solution. In one embodiment, the ascorbic acid is present in an amount of about 8 to about 20 percent by weight of the solution.

In some embodiments, the non-aqueous solutions and slurries of the present invention may be formulated to include boric acid and coconut oil as the dispersing medium. In another embodiment, the non-aqueous solutions and slurries of the present invention may be formulated to include boric acid, coconut oil as the dispersing medium, and vitamin E as an antioxidant. In still another embodiment, the non-aqueous solutions and slurries of the present invention may be formulated to include boric acid, coconut oil as the dispersing medium, and ascorbic acid (vitamin C) as an antioxidant and/or preservative. In yet another embodiment, the non-aqueous solutions and slurries of the present invention may be formulated to include boric acid, coconut oil as the dispersing medium, vitamin E as an antioxidant, and ascorbic acid (vitamin C) as an antioxidant and/or preservative.

The non-aqueous solutions and slurries of the present invention may contain about 100 mg/mL to about 1000 mg/mL weak acid. In one embodiment, the weak acid is included in an amount of about 150 mg/mL to about 900 mg/mL. In another embodiment, the weak acid is included in the non-aqueous solutions and slurries in an amount of about 175 mg/mL to about 800 mg/mL. In yet another embodiment, the non-aqueous solutions and slurries include about 200 mg/mL to about 700 mg/mL of weak acid. In still another embodiment, the weak acid is included in the non-aqueous solutions and slurries in an amount of about 250 mg/ml to about 600 mg/ml. For example, the non-aqueous solutions and slurries may contain about 275 to about 500 mg/ml of boric acid.

In some embodiments, the dispersing media of the non-aqueous solutions and slurries of the present invention may be used in an amount of about 1 mL to about 5 mL. In another embodiment, the dispersing media is used in an amount of about 1.5 ml to about 4.5 ml. In yet another embodiment, the dispersing media is used in an amount about 1.75 ml to about 4 ml. In still another embodiment, the dispersing media is used in an amount of about 1.9 ml to about 3.5 ml. In yet another embodiment, the dispersing media is used in an amount of about 1.95 ml to about 3.0 ml. For example, in one embodiment, the dispersing media of the non-aqueous solutions and slurries of the present invention may be used in amount of about 2 mL.

The pH of the resultant non-aqueous solutions and slurries may be about 7 or below at about 20° C. In another embodiment, the resultant non-aqueous solutions and slurries may have a pH of about 2 to about 7 at about 20° C. In yet another embodiment, the pH of the resultant non-aqueous solutions and slurries is from about 2 to about 6.5. In still another embodiment, the pH of the resultant non-aqueous solutions and slurries is from about 2.5 to about 7. For example, the resultant non-aqueous solutions and slurries may have a pH of from about 2.5 to about 6.5. In yet another embodiment, the pH of the resultant non-aqueous solutions and slurries is from about 4.5 to about 7. In still another embodiment, the pH of the resultant non-aqueous solutions and slurries is from about 5.0 to about 6.

### Powdered Form

In yet other embodiments, the compositions of the present invention may be used in powdered form. For example, the one or more weak acids may be used in the form of a dissolvable powder. In one embodiment, the compositions of the present invention include boric acid in a powdered form. In another embodiment, the compositions of the present invention include propionic acid in a powdered form. In still another embodiment, the compositions of the present invention include carbonic acid in a powdered form.

### Methods of Use

The vaginal compositions of the present invention may be administered to a subject in need of treatment or in need of prevention. The effective amount of the one or more weak acids that a subject receives may vary depending on the specific weak acid(s) employed in the vaginal composition, the severity of symptoms, or a combination thereof. In one embodiment, the effective amount of the one or more weak acids in the composition is about 100 mg to about 1000 mg per day. In another embodiment, the effective amount of the one or more weak acids in the composition is about 300 mg to about 900 mg per day. In still another embodiment, the effective amount of the one or more weak acids in the composition is about 500 mg to about 700 mg per day. For example, the effective amount of the one or more weak acids in the composition is about 600 mg per day. In other embodiments, the effective amount of the one or more weak acids in the composition is about 300 mg per day.

The disclosed compositions may be administered to a subject or by a subject with any suitable device that allows the composition to be inserted/applied to the vagina and/or labial area. For example, the compositions described herein, such as the aqueous solutions, may be inserted directly into the vaginal cavity via a device, such as a bellows bottle, that allows the solution to flow into the vaginal cavity. In this aspect, the subject may insert the device while sitting or standing approximately two to four inches into the vagina. In one embodiment, the vaginal device allows at least 80 percent of the solution to flow into the vaginal cavity. In another embodiment, the vaginal device allows at least 85 percent of the solution to flow into the vaginal cavity. In still another embodiment, the vaginal device allows at least 90 percent of the solution to flow into the vaginal cavity. In yet another embodiment, the vaginal device allows at least 95 percent of the solution to flow into the vaginal cavity

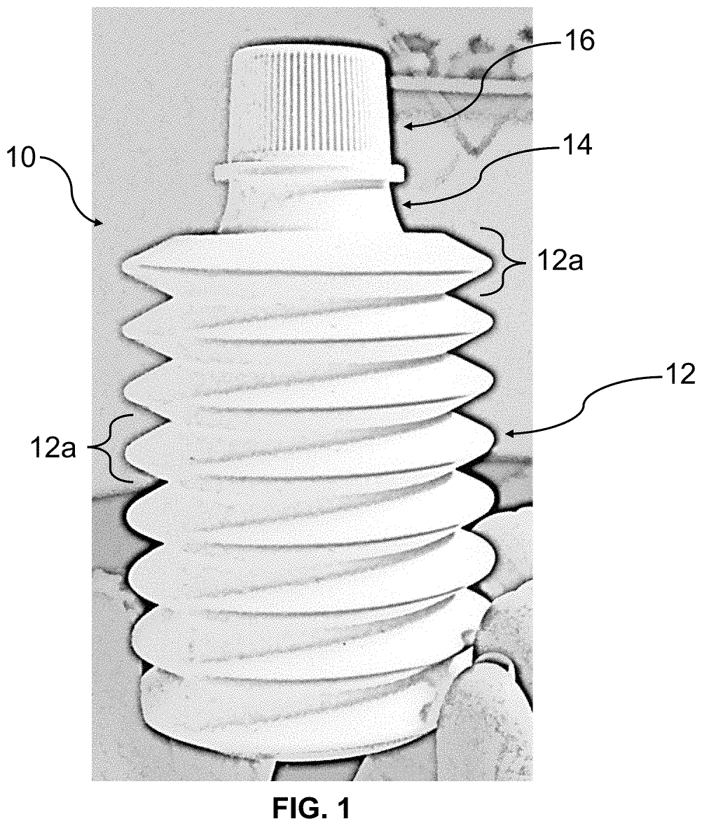

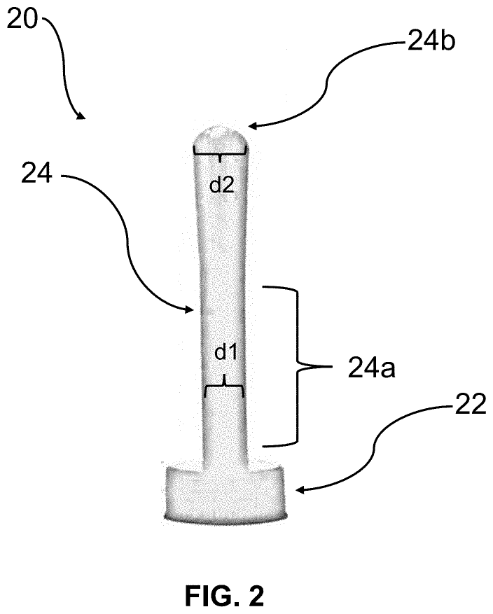

FIG. 1 shows an exemplary vaginal device 10 of the present invention. As shown in FIG. 1, the device 10 has a storage portion 12 that includes multiple accordion-like collapsible bellows 12a, a neck 14, and a threaded collar (not shown) to receive a storage cap 16 and, alternatively, a nozzle 20 as shown in FIG. 2. In the illustrated embodiment, the storage portion 12 includes seven collapsible bellows 12a. However, the storage portion 12 may include any number of bellows, such as between six and nine bellows. The storage portion 12 may be flexible and may be constructed such that it is able to store about 50 mL to about 150 mL of the aqueous solution. In one embodiment, the storage portion 12 is capable of receiving a volume of solution of about 75 mL to about 100 mL. In another embodiment, the storage portion 12 is capable of receiving a volume of solution of about 80 mL to about 90 mL.

The nozzle 20 may be removably attached to the storage portion 12 of the device 10 via threads on the inside of the lower portion 22. The nozzle 20 may also include an insertion portion 24 that includes a middle portion 24a having a first diameter d1 and a top portion 24b having a second diameter d2 and a rounded end with at least one opening to allow the vaginal solution to be expressed during administration. In one embodiment, the first diameter d1 is less than the second diameter d2 by at least about 10 percent. In another embodiment, the first diameter d1 is at least about 20 percent less than the second diameter d2.

In other embodiments, the disclosed compositions may be formulated for transmucosal administration. Transmucosal administration refers to a route of administration in which the composition is diffused through the mucous membrane. For instance, the disclosed compositions may be formulated for vaginal transmucosal administration. In this embodiment, the compositions of the present invention may be incorporated into, for example, a capsule, tablet, suppository, douche, lubricant, tampon, or vaginal ring.

In one embodiment, the compositions of the present invention, for example, the non-aqueous solutions and slurries and the powdered form, may be administered in the form of a vaginal capsule. The vaginal capsule may be made from a number of suitable materials including, but not limited to, hard gelatin (derived from acid-treated raw materials or alkali-treated raw materials), hydroxyl propyl methyl cellulose (HPMC), pullulan, cellulose ethers, such as starches (e.g., waxy maize starch, tapioca dextrin, corn, potato, and derivatives thereof), carrageenan, and polymers or copolymers of (meth)acrylic acids and derivatives thereof. In one embodiment, the capsule is a gelatin capsule. In another embodiment, the capsule is made from pullulan.

The capsule should be formulated to allow for stability during storage and quick disintegration when administered into vagina. In one embodiment, the capsule is made of a material that allows for dissolution in the vagina within about 2 minutes to about 15 minutes. In another embodiment, the capsule is made of a material that allows for dissolution in the vagina within about 4 minutes to about 12 minutes. In still another embodiment, the capsule is made of a material that allows for dissolution in the vagina within about 5 minutes to about 10 minutes.

In other embodiments, the compositions of the present invention, for example, the non-aqueous solutions and slurries and the powdered form, may be administered in the form of a vaginal suppository. In this aspect, the suppository may be torpedo or bullet shaped, round, an elongated oval, teardrop shaped, or cone shaped. The vaginal suppository may be lipophilic based or hydrophilic based. In this regard, vaginal suppositories for use with the present invention may be made of cocoa butter, coconut oil, glycerinated gelatin, hydrogenated vegetable oils and hard fats, polyethylene glycols (PEGs), fatty acid esters of PEG, and combinations thereof. In one embodiment, such as with lipophilic fat-based suppositories, the vaginal suppository melts at body temperature to release the composition of the invention to the body. Without being bound to any particular theory, lipophilic fat-based suppositories may be most useful in connection with delivering the compositions of the invention when vaginal dryness is an issue. In contrast, since the hydrophilic water-based suppositories are unaffected by body temperature and use the body fluids to dissolve the suppository to release the composition to the body, such suppository may be most useful when vaginal dryness is not an issue. In still another embodiment, the compositions of the present invention, for example, the non-aqueous solutions and slurries and the powdered form, may be administered in the form of a vaginal tablet.

Controlled release formulations, for example, delayed release or extended release formulations, may also be desirable. For example, the disclosed compositions, for example, the non-aqueous solutions and slurries and the powdered form, may be coated with one or more controlled release coatings (for example, delayed release or extended release coatings) prior to incorporation into the finished dosage form. In other embodiments, the disclosed compositions, for example, the non-aqueous solutions and slurries and the powdered form, may be incorporated into, for example, a capsule, tablet, suppository, douche, lubricant, tampon, or vaginal ring that is coated with one or more controlled release coatings (for example, delayed release or extended release coatings).

The disclosed compositions may be administered by with or without a suitable applicator. In this aspect, the capsule, tablet, or suppository may be inserted into the vagina manually. Alternatively, the capsule, tablet, or suppository may be supplied as a component of a kit, which includes an applicator. In particular, the capsule, tablet, or suppository may be pre-packaged in the applicator, or supplied as a separate component of the kit.

The disclosed compositions may be administered to the subject on a daily basis until the symptoms are alleviated. For example, the disclosed compositions may be administered to the subject once a day for at least 3-4 days. In another embodiment, the disclosed compositions are administered to the subject at least once a week. In yet another embodiment, the disclosed compositions are administered once a day for at least seven consecutive days. In still another embodiment, the disclosed compositions are administered 3-4 times per week, such as on alternate days.

A normal vaginal pH is in the acidic range (about 3.8 to about 4.5), but that can change with menstruation (pH ranges form about 7 to 7.5), menopause (pH of about 5.3), sexual activity (pH of about 7-8), and even sweat-inducing workouts. As such, the solutions of the present invention may be used in a subject experiencing any of these pH-altering events or conditions on an as-needed basis or regular basis (depending on the specific event or condition). Indeed, it is surprising and unexpected that the compositions of the present invention are capable of altering an elevated vaginal pH such that the pH after administration is lowered. In fact, as would be appreciated by those of ordinary skill in the art, the introduction of a weak acid having a high pKa value to a vagina with an elevated pH (e.g., greater than 4.5) would not, based on standard pH equations like the Henderson-Hasselbalch equation (pH=pKa+log([A⁻]/[HA]) where A⁻ is the concentration of buffer and HA is the concentration of acid) and the Sorensen equation (pH=−log[H₃O⁺]), be expected to lower the vaginal pH. For example, given that the pKa value of boric acid is in the range of 9.14 (at 25° C.) and the pH is in the range of 5.1, the introduction of boric acid to a vagina having a pH of 5 or more would not be expected to reduce the vaginal pH to lower than 5. Rather, it would be expected that the boric acid alone (without a conjugated salt) would adjust the vaginal pH to a range of about 8-10 (based on the Henderson-Hasselbalch equation). However, as shown later in the examples, after administration of the compositions of the invention, the previously elevated vaginal pH is reduced.

In some embodiments, the administration occurs within proximity to sexual activity such as sexual intercourse. In this aspect, the solution may be administered within 24 hours prior to or after intercourse. In one embodiment, the solution is administered within 12 hours prior to or after intercourse. In another embodiment, the solution is administered within 3-4 hours prior to or after intercourse. In other embodiments, the administration occurs before, during, or after menstruation. In this aspect, the solution may be administered within 24 hours after the end of menstruation. In one embodiment, the solution is administered for 1-5 days after the end of menstruation. In yet another embodiment, the solution may be administered during menopause.

One embodiment provides a method of treatment and/or reduction of vaginal odor in a subject. The method may include administering to a subject in need of treatment or in need of prevention an effective amount of the disclosed compositions. In this aspect, the disclosed compositions are effective to neutralize the odiferous compounds in the vagina, such as amines that are produced in the vagina as a result of bacterial activity. The odor of the amines is known to increase at higher pH's because unprotonated, volatile amines are present in basic conditions. Without being bound by any particular theory, it is contemplated that the acidic environment produced by the weak acids utilized in the disclosed compositions causes the odor-producing amines to be protonated, and thereby stabilized in a non-odiferous amide form, or at least their release impeded, further eliminating vaginal odor and neutralizing their odor carrying capabilities. In addition, the solution restores the vagina to the natural pH to below 5, preferably below 4.7, more preferably below 4.5, and even more preferably to a normal vaginal pH (around 4), which reduces the odor associated with the previously abnormal pH.

In another embodiment, the present invention provides a method for adjusting the pH of a subject's vagina, for example, adjusting the pH of a mammalian or human vagina. The methods of the present invention unexpectedly reduce abnormal (elevated) vaginal pH values to normal levels. For example, based on the standard Henderson-Hasselbalch equation and given that the weak acids of the present invention have high pKa values, it would be expected that administration of a weak acid, such as boric acid, to the vagina would increase an abnormally elevated vaginal pH to an even higher pH level (for instance, to a pH in the range of about 8 to 10). Surprisingly, however, administration of the compositions of the present invention are able to lower an elevated vaginal pH to a natural vaginal pH (for example, below 5.0).

In this embodiment, the subject is in need of reduction of an abnormal vaginal pH. The method may include administering an effective amount of any of the disclosed compositions to the subject's vagina. For example, the disclosed compositions may be administered to the subject's vagina such that the disclosed compositions contact the surface of the vaginal epithelial cells. The disclosed compositions are unexpectedly effective in reducing the abnormal vaginal pH to a normal vaginal pH. For instance, in one embodiment, the disclosed compositions are effective to reduce an abnormal vaginal pH to a natural pH of about 5.0 or below. In another embodiment, the disclosed compositions are effective to reduce an abnormal vaginal pH to a natural pH of about 4.7 or below. In still another embodiment, the disclosed compositions are effective to reduce an abnormal vaginal pH to a natural pH of about 4.5 or below. In yet another embodiment, the disclosed compositions are effective to reduce an abnormal vaginal pH to a natural pH of about 4.0.

In still other embodiments, the disclosed compositions are effective to reduce the vaginal pH of a subject by about 0.5. In another embodiment, the disclosed compositions are effective to reduce the vaginal pH of a subject by about 1.0. In still another embodiment, the disclosed compositions are effective to reduce the vaginal pH of a subject by about 1.5. In yet another embodiment, the disclosed compositions are effective to reduce the vaginal pH of a subject by about 2.0.

The reduction in pH of a subject's vagina advantageously reduces the odor associated with an abnormally high vaginal pH. As discussed above, it is believed that the disclosed compositions are effective to cause protonation of the odoriferous amines within the subject's vagina, thereby eliminating or reducing vaginal odor. In some embodiments, after administration of the disclosed compositions, a subject may experience a negative whiff test within about 24 hours or less. A “negative whiff test,” as used herein, refers to a negative result obtained after performance of a whiff test in which potassium hydroxide is added to vaginal fluid for assessment of amine odor. In another embodiment, after administration of the disclosed compositions, a subject may experience a negative whiff test within about 12 hours or less. In still another embodiment, after administration of the disclosed compositions, a subject may experience a negative whiff test within about 8 hours or less. In yet another embodiment, after administration of the disclosed compositions, a subject may experience a negative whiff test within about 4 hours or less.

In another embodiment, the compositions are administered to a subject experiencing itchiness or irritation in and/or around the vagina. In this aspect, the method may include administering to a subject in need of treatment or in need of prevention an effective amount of the disclosed compositions such that the symptoms are alleviated within about 12 hours or less. In another embodiment, the symptoms are alleviated within about 8 hours or less. In yet another embodiment, the symptoms are alleviated within about 4 hours or less. In some embodiments, the method includes administering the disclosed compositions including a moisturizing agent, such as coconut oil or vitamin E, to a subject experiencing itchiness or irritation in and/or around the vagina.

In one embodiment, administration of the vaginal compositions treats or prevents at least one symptom of a vaginal infection, condition, or disturbance. For example, the administration of the compositions alleviates one or more symptoms of vaginal candidiasis, bacterial vaginosis (BV), and/or trichomonal, such as vaginal discharge, vaginal pH, malodor, positive whiff test, or presence of clue cells. In another embodiment, two or more such symptoms are alleviated. In still another embodiment, three or more such symptoms are alleviated. For instance, the administration of the compositions alleviates at least three of the four Amsel's criteria used in diagnosing BV. In other embodiments, administration of the vaginal compositions of the present invention reduces the Nugent score of a subject. For example, a subject having a Nugent score of 4 to 10 pre-administration can be treated with the compositions of the present invention to reduce the subject's Nugent score to a normal level, i.e., 3 or below.

In some embodiments, the effect of the solution on a subject is compared to a control. For example, the effect of the solution on a particular symptom can be compared to an untreated subject or the condition of the subject prior to treatment. In some embodiments, the symptom is recorded or measured in a subject prior to treatment, and again one or more times after administration. In some embodiments, the effect of the treatment is compared to a conventional treatment that is known in the art.

In yet another embodiment, the disclosed solution is administered in the disclosed device to a subject with vaginitis as a combination treatment with a conventional pharmaceutical like metronidazole, clindamycin, and tinidazole. For instance, in one embodiment, one or symptoms of vaginal infection, such as vaginal itching, vaginal irritation, and vaginal odor, are alleviated within about 24 hours after administration of the disclosed solution. In another embodiment, the one or more symptoms of the vaginal infection are alleviated within about 12 hours after administration. In still another embodiment, the one or more symptoms of the vaginal infection are alleviated within about 8 hours after administration. In yet another embodiment, the one or more symptoms of the vaginal infection are alleviated within about 4 hours after administration.

The disclosed solutions can be administered to a subject in need thereof in combination or alternation with other therapies and therapeutic agents. In some embodiments, the disclosed solutions and the additional therapeutic agent are administered separately, but simultaneously, or in alternation. The disclosed solutions and the additional therapeutic agent can also be administered as part of the same composition. In other embodiments, the disclosed solutions and the additional therapeutic agent are administered separately and at different times, but as part of the same treatment regime. Suitable therapeutic agents contemplated for use in this aspect of the invention include, but are not limited to, anti-inflammatory agents, antifungals, antibiotics, antivirals (especially protease inhibitors alone or in combination with nucleosides for treatment of HIV or Hepatitis B or C), anti-parasites (helminths, protozoans), hormones, and combinations thereof.

### Examples

The following non-limiting examples are merely illustrative of the preferred embodiments of the present invention, and are not to be construed as limiting the invention, the scope of which is defined by the appended claims.

### Example 1: Treatment with Vaginal Suppository of Non-Aqueous Boric Acid Slurry Eliminated Vaginal Odor and Decreased Vaginal pH

A non-aqueous slurry according to the present invention was prepared as a vaginal suppository. The non-aqueous slurry was prepared as follows:
67 mg of vitamin E
25 mg of vitamin C
600 mg of boric acid
2 mL of coconut oil
   A total of 10 subjects presenting with vaginal odor were identified. The subjects were treated with the vaginal suppository containing the non-aqueous slurry. Each of the subjects was assessed for adverse reactions and symptom expression, for example, expression of vaginal odor, after treatment with the vaginal suppository.

After evaluation, it was determined that 100% of the subjects experienced no discomfort or adverse reactions after treatment. It was also determined that 87.5% of the subjects' vaginal odor issues were resolved after treatment with the suppository.

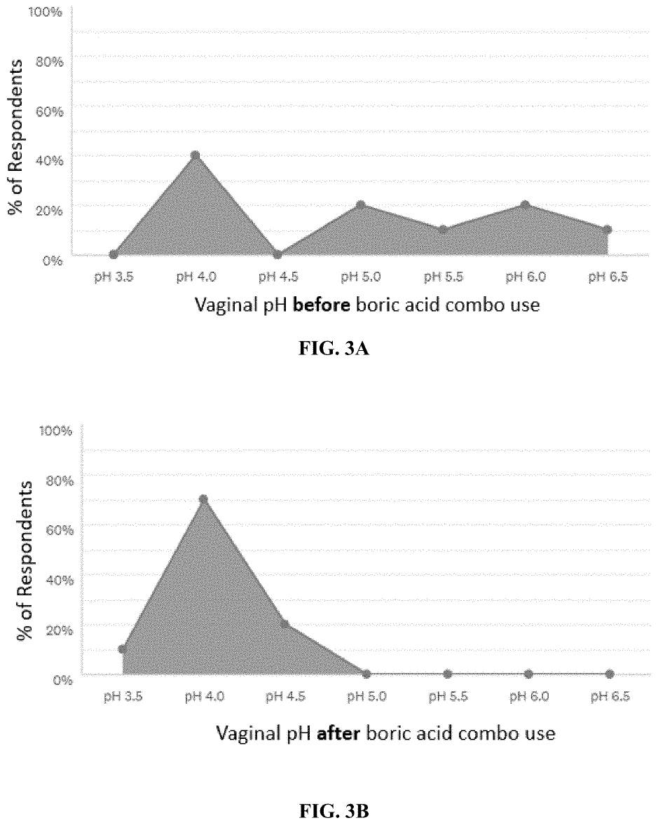

Moreover, treatment with the vaginal suppository resulted in a decreased vaginal pH. FIGS. 3A and 3B show the percentage of subjects having a certain vaginal pH before treatment with the vaginal suppository and after treatment with the vaginal suppository, respectively. As shown by FIGS. 3A and 3B, after the use of the boric acid vaginal suppository, the data skews to the left, displaying an overall more acidic vaginal atmosphere after its use. The reduction in the vaginal pH of the subjects after administration of the vaginal suppository is surprising and unexpected. Indeed, based on the expected pH dynamics of a weak acid with a high pKa, the actual pH of the vagina after administration was not expected based on the Henderson-Hasselbalch equation.

### Example 2: Treatment with Vaginal Suppository of Boric Acid Decreased Vaginal pH

A composition according to the present invention was prepared as a vaginal gelatin suppository. The composition was prepared as follows:
600 mg of boric acid
   A total of 10 subjects were treated with the vaginal suppository containing the composition. The subjects were instructed to check their vaginal pH using pH strips and record the value. The subjects then inserted a single vaginal suppository before bed. The subjects checked their vaginal pH 6 to 8 hours after insertion of the suppository.

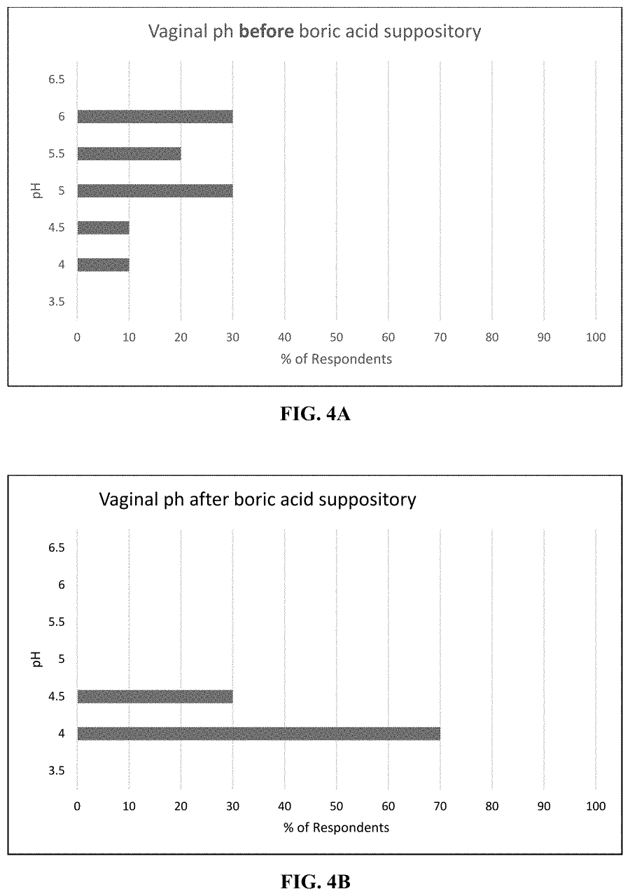

Table 1 below shows the vaginal pH before and after insertion of the vaginal suppository for each subject. FIGS. 4A and 4B show the percentage of subjects having a certain vaginal pH before treatment with the vaginal suppository and after treatment with the vaginal suppository, respectively.

TABLE 1
Vaginal pH of subjects before and after insertion
of the vaginal suppository

| | Vaginal pH before | Vaginal pH after |
|---|---|---|
| Subject | using suppository | using suppository |
| 1 | 4.0 | 4.0 |
| 2 | 4.5 | 4.0 |
| 3 | 5.5 | 4.5 |
| 4 | 5.0 | 4.0 |
| 5 | 5.0 | 4.0 |
| 6 | 5.0 | 4.5 |
| 7 | 6.0 | 4.5 |
| 8 | 5.5 | 4.0 |
| 9 | 6.0 | 4.0 |
| 10 | 6.0 | 4.0 |

As can be seen from Table 1 and FIGS. 4A and 4B, treatment with the vaginal suppository resulted in a decreased vaginal pH for 90% of the subjects. The reduction in the vaginal pH of the subjects after administration of the vaginal suppository is surprising and unexpected. Indeed, based on the expected pH dynamics of a weak acid with a high pKa, the actual pH of the vagina after administration was not expected based on the Henderson-Hasselbalch equation.

### Prophetic Example 3: Vaginal Douche of Aqueous Boric Acid Solution

A suitable formulation for a solution according to the present invention for the treatment of vaginal irritation and odor may be as follows, wherein the percentages are given as percent by weight based on the total weight of the composition: 1-5 percent boric acid, 99-95 percent water.

Other ingredients may be added and include those typically found in vaginal douches such as other antimicrobial agents, anesthetics, or antipruritics (such as phenol or menthol), astringents and surface active agents. The solution may be initially formed as a concentrated liquid, dissolvable powder or tablet. When use is desired, water may be added, preferably warm in temperature, to produce a solution of desired concentration.

### Prophetic Example 4: Treatment with Vaginal Douche of Aqueous Boric Acid Solution Alleviates Vaginal Itching and Vaginal Odor

Subjects presenting with vaginal odor and itch symptoms are identified. The subjects are treated using the vaginal douche in Prophetic Example 1. A total of 60 subjects participate in the study: 30 subjects are treated with the vaginal douche in Prophetic Example 1 (Group 1) and 30 subjects are treated with a vinegar and water douche (Group 2). Each of the subjects are evaluated with a pH test and assessed for symptom expression after the first day of treatment, after the third day of treatment, after the fifth day of treatment, and after the seventh day of treatment.

Within about 12 hours to 24 hours after administration of the vaginal douche, each of the subjects in Group 1 experience alleviation of vaginal itching, vaginal odor, or both.

### Prophetic Example 5: Vaginal Capsule of Non-Aqueous Boric Acid Solution

A suitable formulation for a solution according to the present invention for the treatment of vaginal irritation and odor may be as follows, wherein the percentages are given as percent by weight based on the total weight of the composition: 100-600 mg boric acid, 25-400 mg vitamin E (100-200 IU), and 0.25-2 ml coconut oil.

The solution may be contained in a gelatin capsule. When use is desired, water may be added, preferably warm in temperature, to produce a solution of desired concentration.

### Prophetic Example 6: Treatment with Vaginal Capsule of Non-Aqueous Boric Acid Solution Alleviates Vaginal Itching and Vaginal Odor

Subjects presenting with vaginal odor and itch symptoms are identified. The subjects are treated using the capsule in Prophetic Example 1. A total of 10 subjects participate in the study: 5 subjects are treated with the capsule in Prophetic Example 1 (Group 1) and 5 subjects are treated with a vaginal douche including vinegar and water (Group 2). Each of the subjects are evaluated with a pH test and assessed for symptom expression after the first day of treatment, after the third day of treatment, after the fifth day of treatment, and after the seventh day of treatment.

Within about 12 hours to 24 hours after administration of the capsule, each of the subjects in Group 1 experience alleviation of vaginal itching, vaginal odor, or both.

It is to be understood that any given elements of the disclosed embodiments of the invention may be embodied in a single structure, a single step, a single substance, or the like. Similarly, a given element of the disclosed embodiment may be embodied in multiple structures, steps, substances, or the like.

The foregoing description illustrates and describes the processes, machines, manufactures, compositions of matter, and other teachings of the present disclosure. Additionally, the disclosure shows and describes only certain embodiments of the processes, machines, manufactures, compositions of matter, and other teachings disclosed, but, as mentioned above, it is to be understood that the teachings of the present disclosure are capable of use in various other combinations, modifications, and environments and are capable of changes or modifications within the scope of the teachings as expressed herein, commensurate with the skill and/or knowledge of a person having ordinary skill in the relevant art. The embodiments described hereinabove are further intended to explain certain best modes known of practicing the processes, machines, manufactures, compositions of matter, and other teachings of the present disclosure and to enable others skilled in the art to utilize the teachings of the present disclosure in such, or other, embodiments and with the various modifications required by the particular applications or uses. Accordingly, the processes, machines, manufactures, compositions of matter, and other teachings of the present disclosure are not intended to limit the exact embodiments and examples disclosed herein. Any section headings herein are provided only for consistency with the suggestions of 37 C.F.R. § 1.77 or otherwise to provide organizational queues. These headings shall not limit or characterize the invention(s) set forth herein.

What is claimed is:

1. A method of adjusting pH in a mammalian vagina, comprising:
administering an effective amount of a composition to a mammalian vagina having a first pH greater than about 4.5, wherein the composition comprises a weak acid having a pH of about 3 to about 7, and wherein the effective amount is sufficient to reduce the first pH to a second pH of about 4.5 or less.

2. The method of claim 1, wherein the mammalian vagina is a human vagina.

3. The method of claim 1, wherein the composition is formulated as an aqueous solution comprising the weak acid uniformly distributed in a solvent comprising water.

4. The method of claim 1, wherein the composition is formulated as a slurry comprising the weak acid dispersed in a dispersing medium comprising cocoa butter, cottonseed oil, lipid nanoparticles, coconut oil, palm oil, palm kernel oil, apricot kernel oil, argan oil, baobab seed oil, calendula oil, grapeseed oil, jojoba oil, sesame seed oil, shea butter, sunflower oil, almond oil, wheat germ oil, or combinations thereof.

5. The method of claim 4, wherein the slurry is administered to the mammalian vagina in the form of a vaginal capsule or vaginal suppository.

6. The method of claim 1, wherein the weak acid is in powder form.

7. The method of claim 6, wherein the weak acid is administered to the mammalian vagina in the form of a vaginal capsule or vaginal suppository.

8. The method of claim 1, wherein the effective amount is sufficient to reduce the first pH to a second pH that is at least about 0.5 lower than the first pH.

9. The method of claim 1, wherein the effective amount is sufficient to reduce the first pH to a second pH that is at least about 1.0 lower than the first pH.

10. A method of adjusting pH in a mammalian vagina, comprising:
administering an effective amount of a composition to a mammalian vagina having a first pH greater than about 4.5 and comprising odoriferous amines, wherein the composition comprises boric acid, and wherein the effective amount is sufficient to (i) reduce the first pH to a second pH of about 4.5 or less and (ii) cause protonation of the odoriferous amines.

11. The method of claim 10, wherein the mammalian vagina is a human vagina.

12. The method of claim 10, wherein the composition is formulated as a slurry comprising the boric acid dispersed in a dispersing medium comprising coconut oil.

13. The method of claim 10, wherein the composition is formulated as an aqueous solution comprising the boric acid uniformly distributed in water.

14. The method of claim 10, wherein the effective amount is sufficient to reduce the first pH to a second pH of about 4.0.

15. The method of claim 10, wherein the effective amount is sufficient to reduce the first pH to a second pH that is at least about 2.0 lower than the first pH.

16. A method of adjusting pH in a mammalian vagina, comprising:
administering an effective amount of a composition to a mammalian vagina having a first pH greater than about 4.5, wherein the composition comprises a weak acid having a pKa of about 7 to about 9.5, and wherein the effective amount is sufficient to reduce the first pH to a second pH of about 4.5 or less.

17. The method of claim 16, wherein the weak acid is selected from the group consisting of boric acid, hydrogen sulfide, ethanoic/acetic acid, lactic acid, citric acid, tartaric acid, ascorbic acid, maleic acid, propionic acid, carbonic acid, and combinations thereof.

18. The method of claim 16, wherein the effective amount is sufficient to reduce the first pH to a second pH of about 4.0.

19. The method of claim 16, wherein the composition is formulated as a slurry comprising the weak acid dispersed in a dispersing medium comprising cocoa butter, cottonseed oil, lipid nanoparticles, coconut oil, palm oil, palm kernel oil, apricot kernel oil, argan oil, baobab seed oil, calendula oil, grapeseed oil, jojoba oil, sesame seed oil, shea butter, sunflower oil, almond oil, wheat germ oil, or combinations thereof.

20. The method of claim 16, wherein the weak acid is in powder form.
